(19) United States
(12) Patent Application Publication
(10) Pub. No.: US 2026/0165938 A1
(43) Pub. Date: Jun. 18, 2026

(54) COSMETIC METHOD AND USE OF A PREPARATION FOR IMPROVING SKIN CHARACTERISTICS

(71) Applicants: Tamás RÓZSA, Mogyoród (HU); Zoltán Szabolcs LÁDI, Budapest (HU)

(72) Inventors: Tamás RÓZSA, Mogyoród (HU); Zoltán Szabolcs LÁDI, Budapest (HU)

(21) Appl. No.: 19/418,744

(22) Filed: Dec. 12, 2025

(30) Foreign Application Priority Data
Dec. 18, 2024 (HU) P2400584

(51) Int. Cl. A61K 8/64; A61K 8/22; A61K 8/73; A61N 5/06; A61N 5/067; A61Q 19/00; A61Q 19/02; A61Q 19/08

(52) U.S. Cl. CPC A61K 8/64; A61K 8/22; A61K 8/73; A61N 5/0616; A61N 5/067; A61Q 19/00; A61Q 19/007; A61Q 19/02; A61Q 19/08; A61K 2800/884; A61N 2005/0659

(57) ABSTRACT

Cosmetic method for improving skin characteristics, in which the cleansed skin surface is sprayed with liquid containing four-atom oxygen, known in itself as activated stabilized oxygen, and then, after the skin has absorbed the spray, the surface is coated with a preparation containing at least 0.040 w % of hydrolyzed beta-glucan, preferably 0.1 w %, wherein the preparation also contains at least 0.04 w %, preferably 0.1 w % acetyl tetrapeptide-40, in the form of a gel or cream, and then the pretreated skin surface is irradiated with soft laser light emitting scattered rays with a wavelength of 600-1000 nm and a power density of 40-200 mW/cm² power density, where the irradiation time is at least 30 seconds, depending on the power density. The composition contains at least 0.040 w % of hydrolyzed beta-glucan, 1-5 w % of a moisturizing agent, 15-20 w % of a skin conditioning agent, 1-5 w % of a preservative, and at least 0.04 w % of acetyl tetrapeptide-40.

### FIELD

The invention relates to a cosmetic method and the use of an associated preparation for improving skin characteristics.

### BACKGROUND

Countless preparations are used in the cosmetics industry that have a beneficial effect on the characteristics of skin (i.e., a number of properties that characterize the condition of the skin, including, for example, skin elasticity, hydration, firmness, redness, wrinkling, number of acne or pimples, presence of rosacea and/or age spots, and reduction in the severity of these conditions, and in many cases, the advertisements for such preparations use the most favorable terms to describe their benefits.

It is a known fact that the human body has mechanisms that trigger the regeneration of skin and tissues, usually in response to injury. In the field of cosmetics, there are a number of treatments known to deliberately cause damage to the skin in order to trigger these regeneration mechanisms. In many cases, these procedures cause serious discomfort to the persons treated, requiring them to endure pain.

There are supposedly “gentle” laser skin regeneration procedures that use a large number of very small, separate infrared laser beams with sufficient energy density to destroy cells. These procedures are often associated with significant discomfort for patients undergoing treatment and require them to endure pain. Such skin regeneration procedures are described, e.g., in U.S. Pat. Nos. 10,245,108B1 and 6,328,733B1.

The natural regeneration process is also influenced by a number of other factors, such as the oxygen supply in the region concerned and the condition of the fibroblasts involved in regeneration. If the conditions necessary for regeneration, or some of them, are not available or are not sufficiently active, then the existence of the aforementioned causes triggering tissue regeneration is not sufficient for the natural regeneration process to take place to a sufficient extent.

There are many types of non-destructive treatments to trigger regeneration of skin and tissues, but are limited in that the absence of all the conditions necessary for natural cell regeneration makes treatment partial and time-limited. Furthermore, most of these treatments are costly and require long-term use. One of the most major reasons for these problems is that the various preparations are only able to reach the deepest layers of the skin to a limited extent, and in addition, the conditions necessary for cell regeneration in aging skin are often only available to a limited extent and cannot be triggered by the methods used.

Cell regeneration can be triggered not only by local damage, but also, for example, by the presence of sufficient amounts of beta-glucan. Kaitlyn McLintock's article “Beta-glucan: a skin care ingredient with great potential,” updated on Apr. 23, 2024, states that beta-glucan is “better than hyaluronic acid.” According to one of the authors, beta-glucan is “a moisturizer that attracts water to the upper layers of the skin,” and, according to feedback from treated individuals, improves skin hydration, reduces wrinkles and redness, and has antioxidant effects.

According to a review published in https:/inci.guide/carbohydrates/hydrolyzed-beta-glucan, beta-glucan belongs to the polysaccharides (consisting of multiple sugars, starch, and cellulose linked together) found in yeast, certain bacteria, fungi, seaweed, and certain grains, such as oat, and then goes on to list the benefits of its use at length. Not all beta-glucans have immunomodulatory effects—only those extracted from yeast and fungi. Beta-glucan extracted from these natural substances has a long molecular length and therefore cannot penetrate the deeper layers of the skin, so its effect is only experienced near the surface. However, the molecule can be broken down by enzymes, and this treatment can be used to produce hydrolyzed beta-glucan. Such a substance is available, among others, in the form of the hydrolyzed beta-glucan product marketed under the trade name Wellmune™ by Kerry Group PLC (Trale, Ireland), whose international INCI name is “hydolyzed beta-glucan” and CAS code is 9051-97-2. This substance retains the aforementioned beneficial effects and, due to its shorter molecular length, can penetrate deeper layers of the skin under certain conditions.

It is known that treatment with soft lasers also has a stimulating effect on living tissues, and a number of laser devices are used for cosmetic purposes, among other things. An effective and high-performance soft laser device is described, for example, in PCT Publication No. WO2019/211638A1, which is expressly incorporated herein by reference. In this description, the term “soft laser” is used to refer to laser devices that do not cause damage to irradiated cells or impair their physiological functions, even when irradiated directly onto the skin surface for a prolonged period, i.e., more than 5 minutes. In this sense, the previously mentioned devices, which have a very small diameter but intense beam, and thus coagulate the cells in their path, cannot be considered soft lasers.

The websites www.safelaser.hu and www.safelaser.eu list cosmetic applications among the many beneficial effects of soft lasers on the body and recommend a gel containing hydrolyzed beta-glucan and numerous other ingredients beneficial to the skin. According to the explanation, hydrolyzed beta-glucan activates Langerhans cells in the lower layers of the skin, triggering a regeneration process, and laser light (in the recommended infrared range) stimulates this process. Of course, this refers to the hydrolyzed beta-glucan mentioned in the aforementioned website. Laser light helps beta-glucan penetrate the layers of the skin.

In a completely different field, to increase the strength of athletes and weakened individuals, the American company Oxygenesis Inc. (Paso Robles, CA, USA) has developed an ASO™ (activated stabilized oxygen) product, the characteristics and recommended uses of which can be found on the company's website: https://www.oxigenesisinc.com/products. This product is a consumable liquid containing large amounts of oxygen in O4 form that can be utilized by the body, where the four-atom oxygen breaks down into two-atom oxygen when used.

Skin redness, rosacea, and skin color-related problems and their treatment are comprehensively described in the article “The Pathophysiology of Melanin Pigmentation” by Dr. Éva Remenyik and colleagues (Börgyógáeszati és Venerológiai Szemle [Dermatology and Venereology Review], vol. 81, no. 5, pp. 197-208). The article recommends several preparations for inhibiting melanin pigmentation

An effective agent that treats skin redness is the product marketed under the trade name TELANGYN® by the Spanish company LIPOTEC (Barcelona), marketed under the trade name TELANGYN®, is an effective remedy against skin redness. It is a peptide-containing gel in which the peptide component is a substance called “acetyl-tetrapeptide-40” (INCI name), to which “caprylyl glycol” (also INCI name) is added. The latter has a preservative effect and also conditions the skin. A detailed description of this preparation can be found on the website https://www.lubrizol.com/Personal-Care/Products/Product-Finder/Products-Data/Telangyn-peptide-solution, among others, but the product is also part of numerous cosmetic preparations. The same company has also developed another similar product for cosmetic purposes under the trade name SNAP-8@, in which the peptide ingredient is listed under the INCI name “acetyl octapeptide-3”. This preparation is recommended as a general skin rejuvenating and wrinkle-removing preparation and is included as an ingredient or raw material in the cosmetic offerings of many companies.

There remains a need to create a method and the necessary preparation for it that is suitable for improving the characteristics of the skin (which can be called regeneration or rejuvenation), including increasing skin elasticity, reducing wrinkles, reducing or removing redness, rosacea and acne, and improving a number of other skin characteristics.

### SUMMARY

In accordance with one aspect of the present inventions, a cosmetic method for improving skin characteristics comprises applying (e.g., via spraying) liquid (e.g., water) containing four-atom stabilized oxygen to a skin surface, allowing the skin to absorb the liquid containing four-atom stabilized oxygen, covering the skin surface with a preparation (e.g., a gel or cream) containing hydrolyzed beta-glucan and acetyl tetrapeptide-40, and irradiating the covered skin surface (e.g., for at least 30 seconds) with laser light operating at a wavelength of 600-1000 nm, preferably in the range of 700-900 nm (e.g., in the range of 805-810 nm), thereby improving the characteristics of the skin. In one method, the covered skin surface is irradiated with the laser light at an energy density in the range of 40-200 mW/cm², and preferably at least 60 mW/cm². Another method further comprises cleaning the skin surface prior to applying the water containing four-atom stabilized oxygen to the skin surface. In one method, the preparation contains at least at least 0.040 w % of hydrolyzed beta-glucan and at least 0.040 w % of acetyl tetrapeptide-40. Still another method, irradiating the covered skin surface comprises moving the laser light source along the skin surface and continually irradiating in such a manner that the duration of the irradiation per area lasts at least 10 seconds. The treatment may be repeated one or twice a day for several days (e.g., four weeks). In still another method, the laser light comprises scattered light rays. Yet another method further comprises moisturizing the skin surface prior to covering the skin surface with the preparation.

In accordance with a second aspect of the present inventions, a preparation for cosmetic use of for improving skin characteristics comprises hydrolyzed beta-glucan, acetyl tetrapeptide-40, a skin conditioning agent (e.g., one of panthenol, soluble collagen, glycerin, hydrolyzed hyaluronic acid, or any combination thereof), and a preservative. The amount of the hydrolyzed beta-glucan may be, e.g., at least 0.040% wt %, and an amount of the acetyl tetrapeptide-40 is at least 0.040% wt %. The amount of skin conditioning agent may be, e.g., at least 15-20% wt %. In one embodiment, the preparation may be a gel that further comprises a gelling agent and water. In this embodiment, the amount of gelling agent may be in the range of 1-5 wt %. In another embodiment, the preparation may be a cream that further comprises an emulsifier, an antioxidant, a gelling agent, and water. In this embodiment, the amount of the emulsifier may be, e.g., in the range of 5-10 wt %, the amount of the antioxidant may be, e.g., in the range of 0.1-1 wt %, and the amount of gelling agent may be, e.g., in the range of 0.1-1 wt %. In still another embodiment, the preparation further comprises four-atom stabilized oxygen.

Other and further aspects and features of embodiments of the disclosed inventions will become apparent from the ensuing detailed description in view of the accompanying FIGURES.

### BRIEF DESCRIPTION OF THE DRAWINGS

The drawings illustrate the design and utility of preferred embodiments of the present invention, in which similar elements are referred to by common reference numerals. It should be noted that the figures are not drawn to scale and that elements of similar structures or functions are represented by like reference numerals throughout the figures. It should also be noted that the figures are only intended to facilitate the description of the embodiments. They are not intended as an exhaustive description of the invention or as a limitation on the scope of the invention, which is defined only by the appended claims and their equivalents. In addition, an illustrated embodiment of the disclosed inventions need not have all the aspects or advantages shown. An aspect or an advantage described in conjunction with a particular embodiment of the disclosed inventions is not necessarily limited to that embodiment and can be practiced in any other embodiments even if not so illustrated. In order to better appreciate how the above-recited and the other advantages and objects of the present inventions are obtained, a more particular description of the present inventions briefly described above will be rendered by reference to specific embodiments thereof, which are illustrated in the accompanying drawings. Understanding that these drawings depict only typical embodiments of the invention and are not therefore to be considered limiting of its scope, the invention will be described and explained with additional specificity and detail through the use of the accompanying drawings in which:

FIG. 1 is a flow diagram of an exemplary method of cosmetically treating a skin surface with a preparation in accordance with the present inventions.

### DETAILED DESCRIPTION OF THE ILLUSTRATED EMBODIMENTS

The invention is based, among other things, on the recognition that by exploiting the known properties of hydrolyzed beta-glucan, namely its activating effect on Langerhans cells, the entire process of skin cell regeneration can be stimulated to the maximum extent. The hydrolyzed form of beta-glucan has a much smaller molecule than the original molecule, but retains the stimulating effects of the original molecule. Due to its small size, it passes through the upper layer of the skin and reaches the Langerhans cells and macrophages without damaging the skin. However, its presence provides the skin's immune system with information about the damage, triggering the production of messenger molecules and the synthesis of elastic fibers by fibroblasts. The effect of hydrolyzed beta-glucan described above (synthesis of elastic fibers) requires energy for fibroblasts, which they produce with the help of their energy-producing cell organelles (mitochondria). If the mitochondria of fibroblasts function poorly, the messenger molecules produced by Langerhans cells and macrophages result in a weaker fiber-forming response.

According to the invention, in addition to activating Langerhans cells, we exert a further stimulating effect on the mitochondria of fibroblast cells, which play a key role in regeneration. We have recognized that acetyl-tetrapeptide-40 aids the function of the cell nucleus of fibroblast molecules and its presence enhances its activation. These two effects, namely the effect of hydrolyzed beta-glucan on fibroblast cells via Langerhans cells and the stimulation of fibroblast cell nucleus function by the presence of acetyl-tetrapeptide-40, together accelerate the skin renewal process.

However, cell proliferation requires that the substances necessary for growth are available in sufficient quantities in their environment. One of the conditions for cell function is the presence of oxygen, which is available in sufficient quantities in young skin through the capillaries, but the blood supply to aging skin is already reduced. Thus, the regeneration process in aging skin is generally inhibited or slowed down due to the lack of the necessary amount of oxygen, which manifests itself in reduced mitochondrial function and regeneration not proceeding at the required rate. We compensate for the local oxygen deficiency necessary for cell development by spraying the cleansed skin surface with the O4-containing ASO™ water before applying the substances and waiting a short time after application until the skin absorbs the spray and the O4 molecules reach the epidermis.

A special feature of the oxygen-enriched ASO™ water is that the oxygen formed during the electrolysis process used in its production is not formed in an O2 molecular structure, but rather in an O4 molecule formed by the combination of four oxygen atoms. Unlike O2 molecules, O4 molecules do not escape in gaseous form but remain in solution. Analytical tests show that the concentration of O4 molecules in ASO™ water is 350,000 ppm. This means that out of 1,000,000 molecules, 350,000 are O4 molecules. When this solution is sprayed onto the skin's surface or applied in gel form, the O4 begins to diffuse into deeper layers of the skin, where the O2 concentration has decreased due to poor circulation. Due to the reduced O2 concentration, the O4 molecule breaks down and releases two O2 molecules, which locally increases the oxygen concentration in the epidermal tissue.

Because hydrolyzed beta-glucan and acetyl tetrapeptide-40 substances are short-chain molecules, their penetration through the different layers of the skin is not impeded. Nevertheless, it is advantageous to enhance penetration with additional measures. One of the known conditions for this is that the skin is treated in good condition, i.e., clean and sufficiently hydrated. This can be achieved by regular moisturizing before the recommended treatment, but it is beneficial to treat the treated skin area with a moisturizing cream suitable for the skin type before or after applying the substances mentioned.

The penetration of the active ingredients into the skin is significantly aided if, after application of the substance, the treated skin area is irradiated with laser light in the wavelength range of 600-1000 nm, and more preferably in the near infrared range of 700-900 nm (e.g., in the wavelength range of 805-810 nm), with an energy density of 40-200 mW/cm², preferably at least 50 mW/cm². The upper limit of intensity is determined only by the power density that burns the skin, i.e., is not gentle, but power densities above the aforementioned range do not increase the stimulation effect. The duration of laser irradiation depends on the energy density; it should be at least 30 seconds, but preferably significantly longer, up to 3-5 minutes. Increasing the treatment time beyond this duration has little effect, whereas it is advantageous to repeat the treatment several times (e.g., twice) a day for several days (e.g., four weeks). It is also important that the laser light source does not emit parallel beams, but rather scattered rays, i.e., radiation in all directions at an angle of at least 160° in front of the light source. The effect of scattered light is that if the radiation (even if attenuated) reaches the eye, the scattered light rays can no longer be focused by the lens of the eye onto the sensitive retina, thus preventing the creation of energy densities greater than those permitted. During treatment, it is advisable to close the eyes when treating skin areas close to the eyes.

If the necessary amount of oxygen is provided as described, and therefore sufficient oxygen is available for the mitochondria to function as virtual nanomotors, namely for the combustion of hydrogen molecules derived from sugar, there is still one factor that can limit energy production, namely the inadequate condition of the mitochondria. Unhealthy nutrition, inflammatory factors, the presence of free radicals, and other oxidative stress cause the formation of nitric oxide (NO) in the mitochondria. NO binds to an enzyme called cytochrome C oxidase, which transports oxygen into the mitochondria through the outer membrane. When NO binds to this enzyme, it is unable to transport oxygen into the mitochondria. One effect of infrared laser light is to detach NO from the enzyme, thereby restoring its ability to transport oxygen. Therefore, in addition to the application of the peptide, such laser radiation also influences stimulating mitochondria, which leads to significant improvement in function. Infrared laser light not only enhances the function of fibroblast mitochondria, but also increases their number. The preparation containing the beta-glucan and peptide can be made in the form of gel or cream, and it is beneficial to add other useful components for the skin to these ingredients.

The minimum concentration of the hydrolyzed beta-glucan ingredient, CAS number 9012-72-0, in the final product should be at least 0.040 w % (w %=weight percent), but its upper limit (depending on the packaging from the source of purchase) can reach up to 0.3% w % if it is obtained from a manufacturer's finished product containing additional additives. The preferred upper limit of the range is 0.1 w % (w %=weight percent). Experience shows that concentrations significantly higher than this cannot increase the regeneration effect, so it is only worth defining the effective range with a lower limit. In one embodiment, the hydrolyzed beta-glucan ingredient may be derived from yeast.

The minimum concentration data of acetyl tetrapeptide-40 (relative to the final product) is 0.040 w %, preferably 0.1 w %. If this ingredient is obtained from the previously mentioned Telangyn™ product, it contains the same amount of caprylyl glycol as acetyl tetrapeptide-40, which is 1,2-octanediol, i.e. a diol with the molecular formula CH₍₃₎(CH₍₂₎)_{.5} CHOHCH₂ OH CAS number: 1117-86-8.

In addition to the two active ingredients mentioned above, the recommended formulation is determined by whether we want to prepare it in gel or cream form

In the case of a gel, its composition in w % may be as follows:

| | |
|---|---|
| Gelling agent | 1-5% |
| Moisturizing agent | 1-5% |
| Skin conditioning agent | 15-20% |
| Preservative | 1-5% |
| Solvent | 75-100% |

In the case of a cream composition, its composition in w % may be as follows:

| | |
|---|---|
| Gel-forming agent | 0.1-1% |
| Humectant | 1-5% |
| Skin conditioning agent | 15-20% |
| Preservative | 1-5% |
| Solvent | 75-100% |
| Emulsifier | 5-10% |
| Antioxidant | 0.1-1% |

In both cases, the solvent is water, but it is advantageous if part of the water component is the O4-containing ASO™ water, which, in addition to its previously sprayed application to the skin, provides additional oxygen supply to the lower layers of the skin. As mentioned above, it is advantageous to add additional excipients.

As an example, the composition of the cream that can be used according to the invention is given in Table 1 below. Table 1 lists the English names of the ingredients.

TABLE 1

| Name | INCI name | w % | Supplier | Quality | Function |
|---|---|---|---|---|---|
| WATER | AQUA | Remaining | — | Food | Solvent |
| | | quantity | | | |
| OLIVEM 1000 | CETEARYL | 5.00 | Hallstar | Cosmetic | Emulsifier |
| | OLIVATE, | | | | |
| | SORBITAN | | | | |
| | OLIVATE | | | | |
| CETIOL | UNDECANE | 5.00 | BTC Europe | Cosmetic | Emulsifying |
| ULTIMATE | TRIDECANE | | GmbH | | agent |
| ZEMEA | PROPANEDIOL | 4.00 | Biesterfeld | Cosmetic | Solvent, |
| | | | | | moisturizer, |
| | | | | | humectant |
| DERMOSOFT | GLYCERIN | 4.00 | Evonik Dr. | Cosmetic | Skin |
| 1388 ECO | SODIUM | | Straetmans | | conditioning |
| | LEVULINATE | | GmbH | | agent |
| | SODIUM | | | | |
| | ANISATE | | | | |
| GLYCERIN | GLYCERIN | 3.00 | Alpha | Ph. Eur. | Humectant, |
| | | | Logistic | | skin |
| | | | | | conditioning |
| | | | | | agent |
| CETIOL OE | CAPRYLYL | 3.00 | Biesterfield | Cosmetic | Solvent |
| | GLYCOL | | KFT | | |
| CETEARYL | CETEARYL | 3.00 | Chemiplast | Cosmetic | Emollient, |
| ALCOHOL | ALCOHOL | | Kft. | | emulsifier, |
| | | | | | emulsion |
| | | | | | stabilizer |
| MEADOWFOAM | LIMNANTHES | 3.00 | Natural | Cosmetic | Emollient |
| OIL | ALBA SEED OIL | | Sourcing | | |
| AQUAXYL | XYLITYLGLUCOSIDE; | 1.50 | Seppic | Cosmetic | Moisturizing |
| | ANHYDROXYLITOL, | | | | agent, skin |
| | XYLITOL | | | | conditioning |
| | AQUA | | | | agent |
| | GLUCOSE | | | | |
| CENTELLA | HELIANTHUS | 1.50 | Provital | Cosmetic | Skin |
| ASIATICA OIL | ANNUUS SEED | | | | smoothing |
| | OIL; CENTELLA | | | | agent |
| | ASIATICA | | | | |
| | EXTRACT | | | | |
| DRACORIN CE | GLYCERYL | 1.00 | Biesterfield | Cosmetic | Emollient, |
| | STEARATE | | KFT | | emulsifier, |
| | CITRATE | | | | skin |
| | | | | | conditioner |
| WELLMUNE | HYDROLYZED | 0.09 | Biothere | Cosmetic | Base active |
| BETA GLUCAN | BETA GLUCAN | | | | ingredient |
| TELANGYN | AQUA; ACETYL | 1.00 | Lipotec | Cosmetic | Base active |
| | TETRAPEPTIDE-40; | | | | ingredient |
| | and | | | | |
| | CAPRYLYL | | | | |
| | GLYCOL | | | | |

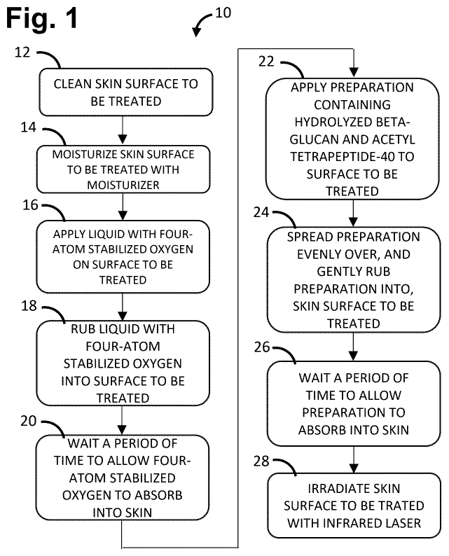

Referring now to FIG. 1, one exemplary method 10 of treating a skin surface according to the present inventions will be described. First, the skin surface to be treated is cleaned (step 12). Next, the skin surface to be treated is moisturized with a moisturizer (e.g., a cream) (step 14). Next, liquid (e.g. water) containing four-atom stabilized oxygen, such as ASO™ O4 is applied (e.g., sprayed) to the cleaned skin surface to be treated (step 16). The ASO™ O4 is then gently rubbed into the still wet skin surface (step 18). The ASO™ O4 is allowed to be absorbed into the skin by waiting a period of time (step 20). Next, a preparation (e.g., gel or cream) containing at least the minimum effective amount of the aforementioned active ingredients, namely hydrolyzed beta-glucan and acetyl tetrapeptide-40, applied to the skin surface to be treated (step 22). The preparation is then spread evenly over, and gently rubbed into, the skin surface to be treated (step 24). The applied gel or cream is allowed to be absorbed into the skin by waiting a period time (step 26).

The skin surface to be treated is then irradiated over a period of time (for at least 10 seconds, but preferably for 1-3 minutes) with in an infrared laser with a wavelength between 600 nm and 1000 nm, and more preferably between 700 nm and 900 nm, and power density of at least 40 mW/cm² (step 28). If the skin surface to be treated is larger than the effective surface of the laser light source, the laser light source may be slowly moved along the skin surface to be treated in such a way that to ensure that each portion of the skin to be treated has at least the required exposure time. For example, the laser light source can be held over one portion of the skin surface to be treated for a duration (e.g., 20 seconds), then moved to another portion of the skin surface to be treated for a duration (e.g., 20 seconds), and then repeated for these portions of the skin surface until both portions of the skin surface has been cumulatively exposed to the laser light for the required time (e.g., one minute). The treatment time is inversely proportional to the power density. For power densities exceeding 100 mW/cm², it may be sufficient to illuminate each area for only about 10 seconds. The method 10 may optionally be repeated (e.g., one or twice a day). The cosmetic effect of the treatment may be illustrated by two examples.

In the first example, the cosmetic effect of the method and composition according to the invention was tested on a group of 50 people (between 31 and 64 year of age, including 7 male volunteers). The kit contained a laser light source with a power of 500 mW, an active surface area of 10 cm² and a wavelength of 809 nm. The participants were also provided with 50 ml bottle of ASO™ water enriched with O4 oxygen, equipped with a spray head, and a cream with the composition listed in Table 1. None of the participants in the study had previously undergone cosmetic or other surgery, nor had they undergone Botox™ or other cosmetic fillers. The participants of the study did not have any identifiable skin diseases. During the study period, they were not allowed to use any skin care products, but the use of make-up was permitted.

The participants performed the treatment twice a day, using the laser for one minute on each skin area for each treatment. Before the test and at the end of the 3-month test period, measurements were taken using devices commonly used for skin examinations. In particular, skin elasticity was tested with a Dual MPA 580 Cutometer device, skin hydration was tested using a Corneometer CM 825 device, and the redness of the skin was examined using a Golorimeter CL-400 device. An average improvement of 71% in skin hydration, a 24% improvement in skin elasticity, and a 21% reduction in skin redness were measured in the participants.

The measurement data are summarized in Table 2 below:

TABLE 2

| Treatment | | Improvement in | Improvement in |
|---|---|---|---|
| number | Age | hydration % | elasticity % |
| 1 | 46 | 67 | 27 |
| 2 | 51 | 81 | 31 |
| 3 | 50 | 71 | 28 |
| 4 | 48 | 75 | 22 |
| 5 | 60 | 67 | 20 |
| 6 | 39 | 69 | 18 |
| 7 | 59 | 72 | 24 |
| 8 | 52 | 80 | 28 |
| 9 | 41 | 76 | 19 |
| 10 | 55 | 73 | 23 |
| 11 | 62 | 73 | 26 |
| 12 | 37 | 77 | 29 |
| 13 | 40 | 65 | 19 |
| 14 | 53 | 75 | 28 |
| 15 | 59 | 69 | 21 |
| 16 | 32 | 73 | 24 |
| 17 | 56 | 70 | 25 |
| 18 | 34 | 78 | 27 |
| 19 | 47 | 67 | 19 |
| 20 | 60 | 73 | 23 |
| 21 | 57 | 79 | 27 |
| 22 | 40 | 69 | 20 |
| 23 | 63 | 71 | 24 |
| 24 | 37 | 72 | 25 |
| 25 | 49 | 75 | 27 |
| 26 | 62 | 70 | 19 |
| 27 | 55 | 76 | 26 |
| 28 | 58 | 65 | 18 |
| 29 | 42 | 70 | 22 |
| 30 | 31 | 69 | 25 |
| 31 | 51 | 73 | 22 |
| 32 | 38 | 76 | 26 |
| 33 | 54 | 71 | 26 |
| 34 | 44 | 67 | 23 |
| 35 | 47 | 71 | 23 |
| 36 | 51 | 70 | 24 |
| 37 | 54 | 73 | 21 |
| 38 | 59 | 76 | 26 |
| 39 | 45 | 68 | 21 |
| 40 | 58 | 74 | 21 |
| 41 | 43 | 75 | 27 |
| 42 | 64 | 67 | 20 |
| 43 | 37 | 76 | 25 |
| 44 | 41 | 72 | 25 |
| 45 | 42 | 73 | 26 |
| 46 | 50 | 69 | 19 |
| 47 | 39 | 72 | 35 |
| 48 | 49 | 69 | 27 |
| 49 | 62 | 70 | 20 |
| 50 | 57 | 76 | 26 |

Significantly, none of the treated participants experienced any significant adverse effects that necessitated discontinuation of treatment for this example.

In the second example, the cosmetic effect of the method and composition according to the invention was tested on a group of 10 people (between 46 and 67 of age). We monitored changes in the skin condition (through the examination of rosacea and melanin (skin pigment spots) of the participants over a period of four weeks. We performed one measurement per week using Colorimeter CL-400 device. The treatment was performing using the procedure and the laser device described in the first example, with the different being that that the treatment time per skin area was, on average, 1 minute and 10 seconds in each case.

During the weekly measurements, we recorded the following average results for rosacea (average values):
1st week: 12% improvement
2nd week: 27% improvement
3rd week: 36% improvement
4^{th} week: 42% improvement

During the weekly measurements, we recorded the following average results for melanin (average values):
1^{st} week: 15.2% improvement
2^{nd} week: 29% improvement
3^{rd} week: 48% improvement
4th week: 64.3% improvement

Significantly, none of the treated participants experienced any significant adverse effects that necessitated discontinuation of treatment for this example.

Examples 1 and 2 confirm that the treatment according to the invention can achieve continuously improving results in as little as four weeks, affecting almost all essential characteristics of the skin, while at the same time not causing any pain or discomfort, and fitting well into people's daily skin care routine. The results also confirmed the initial hypothesis, namely that the effect of hydrolyzed beta-glucan on Langerhans cells, combined with the effect of acetyl tetrapeptide-40, which stimulates the cell nuclei of fibroblasts, can together initiate the skin regeneration process properly, provided that local oxygen supply is ensured and that the scattered laser light not only aids penetration but also stimulates energy production, and thus, the natural function of the cells. It can be assumed that by refining the further details of the treatment and continuing treatment for longer than one month, even more favorable results than the very significant improvement described here can be achieved.

Although particular embodiments of the present inventions have been shown and described, it will be understood that it is not intended to limit the present inventions to the preferred embodiments, and it will be obvious to those skilled in the art that various changes and modifications may be made without departing from the spirit and scope of the present inventions. Thus, the present inventions are intended to cover alternatives, modifications, and equivalents, which may be included within the spirit and scope of the present inventions as defined by the claims.

What is claimed is:

1. A cosmetic method for improving skin characteristics, comprising:
applying liquid containing four-atom stabilized oxygen to a skin surface;
allowing the skin to absorb the liquid containing four-atom stabilized oxygen;
covering the skin surface with a preparation containing hydrolyzed beta-glucan and acetyl tetrapeptide-40; and
irradiating the covered skin surface with laser light operating at a wavelength range of 600-1000 nm², thereby improving the characteristics of the skin.

2. The cosmetic method of claim 1, wherein the wavelength range is 700-900 nm.

3. The cosmetic method of claim 1, wherein the covered skin surface is irradiated with the laser light at an energy density in the range of 40-200 mW/cm.

4. The cosmetic method of claim 3, wherein the wavelength of the laser light is between 805 and 810 nm and the energy density of the laser light is at least 60 mW/cm².

5. The cosmetic method of claim 1, further comprising cleaning the skin surface prior to applying the liquid containing four-atom stabilized oxygen to the skin surface.

6. The cosmetic method of claim 1, wherein applying the liquid containing four-atom stabilized oxygen to the skin surface comprises spraying the liquid containing four-atom stabilized oxygen on the skin surface.

7. The cosmetic method of claim 1, wherein the preparation contains at least 0.040 w % of hydrolyzed beta-glucan and at least 0.040 w % of acetyl tetrapeptide-40.

8. The cosmetic method of claim 1, wherein the irradiation time is at least 30 seconds.

9. The cosmetic method of claim 1, wherein the preparation is in the form of a gel or cream.

10. The cosmetic method of claim 1, wherein the laser light comprises scattered light rays.

11. The method as claimed in claim 1, further comprising moisturizing the skin surface prior to covering the skin surface with the preparation.

12. A preparation for cosmetic use of for improving skin characteristics, comprising:
hydrolyzed beta-glucan;
acetyl tetrapeptide-40;
a skin conditioning agent; and
a preservative.

13. The preparation of claim 12, wherein an amount of the hydrolyzed beta-glucan is at 0.040% wt %, and an amount of the acetyl tetrapeptide-40 is at least 0.040% wt %.

14. The preparation of claim 13, wherein an amount of the skin conditioning agent is at least 15-20% wt %.

15. The preparation of claim 12, wherein the preparation is a gel, and further comprises a gelling agent and water.

16. The preparation of claim 15, wherein the amount of the gelling agent is in the range of 1-5 wt %.

17. The preparation of claim 12, wherein the preparation is a cream, and further comprises an emulsifier, an antioxidant, a gelling agent, and water.

18. The preparation of claim 17, wherein the amount of the emulsifier is in the range of 5-10 wt %, the amount of the antioxidant is in the range of 0.1-1 wt %, and the amount of gelling agent is in the range of 0.1-1 wt %.

19. The preparation of claim 12, further comprising four-atom stabilized oxygen.

20. The preparation of claim 12, wherein the skin conditioning agent is one of panthenol, soluble collagen, glycerin, hydrolyzed hyaluronic acid, or any combination thereof.
